(19) United States
(12) Patent Application Publication
(10) Pub. No.: US 2011/0207165 A1
(43) Pub. Date: Aug. 25, 2011

(54) SMALL SCALE SHAKER FLASK CULTIVATION

(75) Inventors: Jan Kreuzmann, Penzberg (DE); Robert Puskeiler, Meunsing (DE); Caroline Schuster, Peissenberg (DE)

(73) Assignee: Hoffman-La Roche, Inc., Nutley, NJ (US)

(21) Appl. No.: 13/122,360

(22) PCT Filed: Oct. 2, 2009
(86) PCT No.: PCT/EP2009/007069

§ 371 Date: Apr. 1, 2011

(30) Foreign Application Priority Data
Oct. 6, 2008 (EP) 08017475.8

(51) Int. Cl. C12P 21/00; C12N 5/07; C12Q 1/02

(52) U.S. Cl. 435/29; 435/325; 435/69.1; 435/69.6

(57) ABSTRACT

The present invention relates to a method for the cultivation of a mammalian cell in a cultivation medium of a cultivation vessel comprising adjusting the rate of a rotational movement of the cultivation vessel depending on the viable cell density in the cultivation medium.

### CROSS REFERENCE TO RELATED APPLICATIONS

This patent application is made under 35 U.S.C. §371 based on International Application No. PCT/EP2009/007069, filed Oct. 2, 2009 and claims the benefit of priority under 35 USC §119(a) to European patent application number 08017475.8 filed Oct. 6, 2008, the disclosure of which is incorporated herein by reference in their entirety.

### FIELD OF THE INVENTION

The present invention relates to the field of devices for the cultivation of cells.

### BACKGROUND OF THE INVENTION

During the development of biotechnological production processes for producing recombinant polypeptides employing mammalian cells a plurality of parameters have to be adjusted and optimized. Due to the large number of required individual experiments this optimization is done at a small scale, preferably in shaker flasks. But as the geometries and reaction conditions differ between a large scale stirred cultivation vessel and a small scale shaker flask the results obtained at small scale will not represent a true image of the large scale process. These differences have a chemical, biochemical, and process engineering origin.

Therefore it is a general objective to map conditions like cell density, product concentration or product quality as good as possible between the small scale and the large scale processes. In order to make a large scale process and a small scale process as comparable as possible each difference has to be evaluated for its impact on the obtained result. For example, if the large scale process is a stirred process it is advisable to perform the small scale process also as stirred process.

In DE 4415444 it is reported an automated determination system for the on-line determination of the oxygen uptake rate in shaker flasks. Kensy, F., et al. (Biotechnol. Bioeng. 89 (2005)698-708) report oxygen transfer phenomena in 48-well microtiter plates. L-Ascorbic acid regulates growth and metabolism of renal cells is reported by Nowack, G., et al. (Am. Physio. Soc. (1996) C2072-C2080). Randers-Eichhorn, L., et al. (Biotechnol. Bioeng. (1996) 466-477) report noninvasive oxygen measurements and mass transfer considerations in tissue culture flasks. Fluid mixing in shaken bioreactors: Implications for scale-up predictions from microliter-scale microbial and mammalian cell cultures is reported by Micheletti, M., et al. (Chem. Eng. Sci. 61 (2006)2939-2949).

Thus, there is a need to map a large scale stirred mammalian cell cultivation process on a small scale shaker flask and vice versa. Another objective is to provide a small scale shaker flask process in which mechanical stirring for cultivation medium agitation and mixing is realized at small scale.

### SUMMARY OF THE INVENTION

In a first aspect the present invention relates to a method for the cultivation of a mammalian cell in a cultivation medium of a cultivation vessel comprising adjusting the rate of a rotational movement of the cultivation vessel depending:

i) on the viable cell density in the cultivation medium, and/or
ii) on the oxygen partial pressure in the cultivation medium.

In an embodiment, the method according to the invention comprises one or more of the following:
a) cultivating a set of cultivation vessels
   each vessel has a total volume of up to about 200 μl, or
   each vessel has a working volume of up to about 100 μl, or
   each vessel has a total volume of from about 25 ml to about 3000 ml, or
   each vessel has a working volume of about 10 ml to about 1500 ml, and/or
b) agitating the cultivation medium by rotational movement of the entire cultivation vessel,
c) adjusting the pH value of the cultivation medium via the carbon dioxide concentration in the gas phase outside of the cultivation vessel.

In another embodiment the cultivation vessel contains a cultivation medium and an inside gas phase above the cultivation medium. In another embodiment the cultivation vessel contains a membrane or a cap or a lid separating the inside gas phase from the outside gas phase. In one embodiment of the method according to the invention the rotational movement is selected from the group consisting of a simple rotational movement of the entire cultivation vessel, or rotational and rocking movement of the entire cultivation vessel and shaking movement of the entire cultivation vessel.

In a further embodiment, the method according to the invention further comprises:
d) determining the pH value and the pO₂ within the cultivation vessel via a non invasive chemo-optical sensor.

In yet another embodiment, the rate of the rotational movement is adjusted as follows:
i) setting the rate of the rotational movement:
   to about 60 rpm at a cell density of 1×10⁵ cells/ml or lower,
   to about 80 rpm at a cell density of 2.5×10⁵ cells/ml,
   to about 100 rpm at a cell density of 5×10⁵ cells/ml,
   or to a rate linearly or quasi-linearly correlated with the cell density,
ii) increasing the rate of the rotational movement by 20 rpm for each doubling of the viable cell density up to a cell density of 20×10⁵ cells/ml,
iii) increasing the rate of the rotational movement by 20 rpm for each increase of the viable cell density of 20×10⁵ cells/ml up to a cell density of 80×10⁵ cells/ml,
iv) increasing the rate of the rotational movement by 10 rpm for an increase of the viable cell density of 80×10⁵ cells/ml to a cell density of about 100×10⁵ cells/ml.

In one embodiment the rate of the rotational movement is further adjusted as follows:
v) maintaining the rate of the rotational movement at about 200 rpm to about 210 rpm, reducing the mechanical movement speed stepwise to about 135 rpm, and keeping it constant until the cultivation is finished.

In one embodiment in step v) the rate of the rotational movement is maintain at about 200 rpm to about 210 rpm for between about 18 hours and about 30 hours. In another embodiment the rate of the rotational movement in step v) is maintained at about 200 rpm to about 210 rpm for about 24 hours.

One embodiment of the method according to the invention comprises adjusting the rate of the rotational movement depending on the oxygen partial pressure in the cultivation medium in order to maintain the oxygen partial pressure in the cultivation medium at or above a lower threshold level. In one embodiment the rate of the rotational movement is increased in order to increase the oxygen partial pressure in the cultivation medium or it is decreased in order to lower the oxygen partial pressure in the cultivation medium. In one embodiment the change of the rate of the rotational movement to change the oxygen partial pressure is a linear change, or is a polynomial change, or is an exponential change. In one embodiment the oxygen partial pressure lower threshold level is about 25% air saturation.

A second aspect of the present invention is a method for the recombinant production of a heterologous polypeptide comprising the following steps:
a) providing a mammalian cell comprising a nucleic acid encoding the heterologous polypeptide,
b) cultivating said mammalian cell with a method according to the invention, and
c) recovering the heterologous polypeptide from the cultivation medium.

In one embodiment the heterologous polypeptide is an immunoglobulin, or an immunoglobulin fragment, or an immunoglobulin conjugate. In a further embodiment the mammalian cell is a CHO cell, a HEK cell, a BHK cell, a NS0 cell, a SP2/0 cell, or a hybridoma cell.

A third aspect of the present invention is the use of a non invasive chemo-optical sensor for the determination of the pH value and the pO₂ in a small scale cultivation vessel in a method according to the invention.

A fourth aspect of the present invention is the use of a method according to the invention for the determination of the cultivation parameter ranges for a large scale cultivation in a stirred cultivation vessel with a volume of about 1,000 l to about 25,000 l.

### DESCRIPTION OF THE FIGURES

FIG. 1 Course of the pH-value and the pO₂ (oxygen partial pressure) value during the cultivation determined online in the cultivation medium (in situ).

FIG. 2 Comparison of the values for pH and pO₂ between online determination and offline determination.

FIG. 3 Time course of the values for pO₂ (offline and online), the course of the viable cell density and the rate of the rotational movement.

FIG. 4 Comparison of the values for pH between offline and online determination. Additionally the course of the pCO₂ in the incubator is given.

FIG. 5 Exemplary change of the rate of the rotational movement (y-axis) depending on the viable cell density (x-axis) in the cultivation medium.

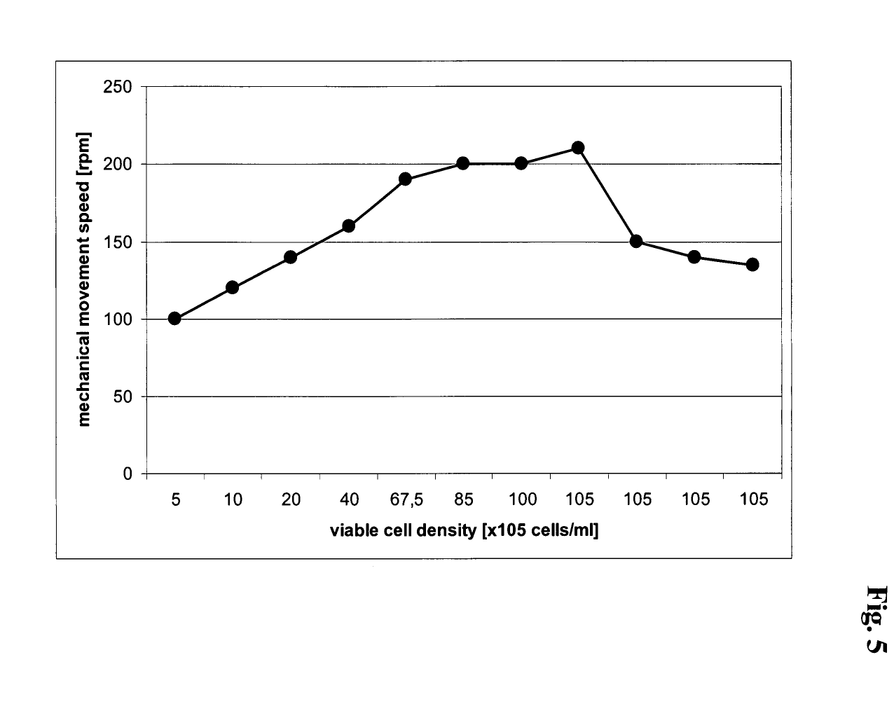

### DETAILED DESCRIPTION OF THE INVENTION

The present invention relates to a method for a small scale shaker flask cultivation of mammalian cells wherein the conditions in the cultivation medium inside the flask are controlled via the rotational rate of the shaker flask and the flask's outside gas pressure.

The present invention is directed to a method for the cultivation of mammalian cells in a shaker flask in which mechanical movement is used for cultivation medium agitation, whereby the pH, pCO₂, and pO₂ are controlled via the rotational rate and the flask's outside gas pressure. The cell density, viability, viable cell density, cell growth curve, volumetric production yield, product concentration, and product quality obtained with a method according to the present invention are comparable to large scale cultivation in a cultivation vessel with mechanical stirring in a total volume of 1,000 l to 25,000 l. Thus, in the method according to the present invention the cultivation medium is not agitated or mixed by a mechanical means, which is in direct contact with the cultivation medium inside the small scale cultivation vessel, such as a mechanical stirring blade or a magnetic stirring bar. The term “mechanical movement” as used herein denotes a way of setting a cultivation medium inside a small scale cultivation vessel in motion, i.e. agitating it or mixing it. The mechanical movement can be as in one embodiment by setting the entire cultivation vessel in motion instead of using a mechanical means that is directly in contact with the cultivation medium inside the cultivation vessel.

A biotechnological production process begins with the cultivation of cells taken from a frozen cell bank as primary cultivation. This primary cultivation is expanded by the transfer of a defined number of cells or defined volume of cultivation medium to a new cultivation vessel which has a larger cultivation volume as that used before and to which fresh additional medium is added. Such cultivations can be performed in cultivation vessels in which the cultivation medium is agitated and mixed by mechanical movement, such as shaking, luffing (rotating and rocking) etc. With a certain cultivation volume it is advantageous to use mechanical stirring for agitating and mixing the cultivation medium, e.g. to minimize concentration differences of cultivation medium components within the cultivation vessel due to insufficient agitation and mixing of the cultivation medium. Such concentration differences may result in non uniform cultivation conditions and, thus, a lack of comparability of cultivation results obtained at different cultivation volumes. Therefore, results obtained and parameters determined at small scale cannot be easily transferred to large scale cultivations without major modifications and, thus, work to be done.

In order to allow a transfer from small scale to large scale some important parameters have been identified in the past:
a geometric comparability of the cultivation vessel, such as ratio of high to diameter or internals like fluid flow breaker (baffles),
a comparable volume specific power input by a comparable dimensioned stirring device,
a comparable mass transport,
a comparable control of important process parameters such as oxygen partial pressure in the cultivation medium, pH value, temperature etc.

A small scale cultivation of mammalian cells according to the present invention has not been reported yet. In this method agitation and mixing of the cultivation medium is by mechanical movement of the entire cultivation vessel. In addition therewith also the control of the partial pressures of oxygen and carbon dioxide and linked thereto the pH value can be achieved. A small scale cultivation when performed with the method according to the present invention results in cell density and product concentration as a large scale cultivation of mammalian cells in a mechanically stirred cultivation vessel. The term “mechanically stirred” or grammatical equivalents thereof as used within the present invention denotes that a mechanical means, e.g. a stirring blade or a stirring bar, is in direct contact with the cultivation medium in the vessel. With a cultivation method as reported herein cultivation parameter ranges for pH value, temperature, mixing speed, carbon dioxide saturation of the gas phase as well as the time-course of the oxygen partial pressure in the cultivation medium can be determined in small scale for the large scale cultivation. The term “range” as used within the present application denotes the spread between the maximum value and the minimum value found for a parameter during a number of small scale cultivations. Therefore, the method according to the invention can be used for the determination of the cultivation parameter ranges for a large scale cultivation in a stirred cultivation vessel with a volume of 1,000 l to 25,000 l by a small scale cultivation in a cultivation vessel of a volume of 25 ml to 3000 ml with a method according to the invention.

In one embodiment the method is with in situ, online determination of the pH-value and the pO₂-value, i.e. the oxygen partial pressure, with a non invasive chemo-optical sensor. The sensors are located in one embodiment in the lower part of the shaker flask and comprise fluorescent, analyte-sensitive dyes embedded in a tissue-compatible polymer which are read out through fiber optics.

The oxygen partial pressure in the liquid cultivation medium can be adjusted by adjustment of the rate of the rotational movement as function of cell density. In this function the rate of the rotational movement can be adjusted stepwise or continuously.

The term “stepwise” as used in the present application denotes a change of a parameter in a cultivation method which is at once, i.e. directly from one value to the next value. In a “stepwise” adjustment is (are) after each change of one or more parameters the changed parameter's value(s) maintained until the next stepwise adjustment in the method or the end of the method.

The term “continuous” as used in the present application denotes a change of a parameter in a cultivation method which is continuous, i.e. the change of the parameter's value is in one embodiment by a sequence of small steps each not bigger than a change of 2%, in another embodiment of 1% of the value of the parameter. In a further embodiment the adjustment is continuous and linear.

It has been found that it is advantageous starting from an inoculation cell density of 5×10⁵ cells/ml with a rate of the rotational movement of 100 rpm to increase this rate by 20 rpm for each doubling of the viable cell density up to a cell density of 20×10⁵ cells/ml and thereafter said rate is increased by 20 rpm for each increase of the viable cell density of 20×10⁵ cells/ml up to a final cell density of 80×10⁵ cells/ml and thereafter said rate is increased by 10 rpm for an increase of the viable cell density of 80×10⁵ cells/ml to 100×10⁵ cells/ml. After reaching a cell density of 100×10⁵ cells/ml the rate is kept constant at increasing viable cell density. In one embodiment the rate of the rotational movement is kept constant at the maximum value for 16 to 72 hours. In another embodiment the rate of the rotational movement is kept constant at the maximum value for 18 hours to 30 hours, in one embodiment for about 24 hours. After the rate of the rotational movement has been kept constant it is reduced stepwise to 135 rpm and is kept constant thereafter until the cultivation is finished.

In more detail, for 5×10⁵ cells/ml the mechanical movement speed is set to 100 rpm (rpm=rounds/rotations per minute), reaching 10×10⁵ cells/ml the rate of the rotational movement is increased to 120 rpm, reaching 20×10⁵ cells/ml the rate of the rotational movement is increased to 140 rpm, reaching 40×10⁵ cells/ml the rate of the rotational movement is increased to 160 rpm, reaching 60×10⁵ cells/ml the rate of the rotational movement is increased to 180 rpm, reaching 80×10⁵ cells/ml the rate of the rotational movement is increased to 200 rpm, reaching 100×10⁵ cells/ml the rate of the rotational movement is increased to 210 rpm. Thereafter the rate of the rotational movement is kept at 200 rpm±20 rpm for about 24 hours. Afterwards the rate of the rotational movement is reduced to 135 rpm. This reduction is performed in one embodiment stepwise, in a different embodiment it is performed continuous. In another embodiment the reduction of the rate of the rotational movement is continuous and asymptotic. After the final rate of the rotational movement of 135 rpm has been reached, the rate of the rotational movement is kept constant until the cultivation is finished and the recombinantly produced polypeptide is harvested.

The changing of the rate of the rotational movement allows to increase or decrease the oxygen partial pressure in the liquid phase depending on the one hand on the viable cell density in the cultivation medium and on the other hand on the phase of the cultivation, i.e. depending on the cultivation being in exponential growth phase, or plateau phase, or production phase.

The term “linear” as used within the present inventions denotes an interrelationship of two parameters that can be expressed as a linear equation, such as y=a*x+b in which y and x denote the two parameters and a and b denote constant values. The term “polynomial” as used within the present inventions denotes an interrelationship of two parameters that can be expressed as a polynomial equation, such as y=a*x+b*x²+c*x³+d*x⁴+ . . . in which y and x denote the two parameters and a, b, c, d and so on denote constant values. The term “exponential” as used within the present inventions denotes an interrelationship of two parameters that can be expressed as an exponential equation, such as y=a*e^{X}+b in which y and x denote the two parameters and a and b denote constant values and e the Euler's number of 2.71828182845904523536.

The adjustment of the pH value in the cultivation medium is in one embodiment by the adjustment of the carbon dioxide partial pressure outside the cultivation vessel. Due to the relationship of gas phase pressure and liquid phase partial pressure, the gaseous carbon dioxide can be forced to enter the liquid phase (pressure rise in the gas phase) or it can be removed from the liquid phase (pressure reduction in the gas phase). Due to the hydration of the carbon dioxide in the liquid cultivation medium, it is a source of hydrogen carbonate and, thus, provides for a carbonate buffer in the liquid phase in order to adjust the pH value of the liquid cultivation medium. In one embodiment of the method according to the invention the pH in the liquid cultivation medium is adjusted via the carbon dioxide gas pressure outside the small scale cultivation vessel, whereby an outside pCO₂ reduction results in a pH increase in the cultivation medium and vice versa.

In one embodiment the cultivating is as a fed-batch cultivation.

A “protein” is a macromolecule comprising one or more polypeptide chains or a polypeptide chain of more than 100 amino acid residues. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

Recombinant production of immunoglobulins is well-known in the state of the art and described, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000)151-161; Werner, R. G., Drug Res. 48 (1998) 870-880.

The term “immunoglobulin” refers to a protein consisting of one or more polypeptide(s) substantially encoded by immunoglobulin genes. The recognized immunoglobulin genes include the different constant region genes as well as the myriad immunoglobulin variable region genes. Immunoglobulins may exist in a variety of formats, including, for example, Fv, Fab, and F(ab)₂ as well as single chains (scFv) or diabodies (e.g. Huston, J. S., et al., Proc. Natl. Acad. Sci. USA 85 (1988) 5879-5883; Bird, R. E., et al., Science 242 (1988) 423-426; in general, Hood et al., Immunology, Benjamin N.Y., 2nd edition (1984); and Hunkapiller, T. and Hood, L., Nature 323 (1986) 15-16).

An immunoglobulin in general comprises two so called light chain polypeptides (light chain) and two so called heavy chain polypeptides (heavy chain). Each of the heavy and light chain polypeptides contains a variable domain (variable region) (generally the amino terminal portion of the polypeptide chain) comprising binding regions that are able to interact with an antigen. Each of the heavy and light chain polypeptides comprises a constant region (generally the carboxyl terminal portion). The constant region of the heavy chain mediates the binding of the antibody i) to cells bearing a Fc gamma receptor (FcγR), such as phagocytic cells, or ii) to cells bearing the neonatal Fc receptor (FcRn) also known as Brambell receptor. It also mediates the binding to some factors including factors of the classical complement system such as component (Clq).

The variable domain of an immunoglobulin's light or heavy chain in turn comprises different segments, i.e. four framework regions (FR) and three hypervariable regions (CDR).

The term “immunoglobulin conjugate” denotes a polypeptide comprising at least one domain of an immunoglobulin heavy or light chain conjugated via a peptide bond to a further polypeptide. The further polypeptide is a non-immunoglobulin peptide, such as a hormone, or growth receptor, or antifusogenic peptide, or complement factor, or the like.

The term “heterologous immunoglobulin” denotes an immunoglobulin which is not naturally produced by a mammalian cell. The immunoglobulin produced according to the method of the invention is produced by recombinant means. Such methods are widely known in the state of the art and comprise protein expression in eukaryotic cells with subsequent recovery and isolation of the heterologous immunoglobulin, and usually purification to a pharmaceutically acceptable purity. For the production, i.e. expression, of an immunoglobulin a nucleic acid encoding the light chain and a nucleic acid encoding the heavy chain are inserted each into an expression cassette by standard methods. Nucleic acids encoding immunoglobulin light and heavy chains are readily isolated and sequenced using conventional procedures. Hybridoma or B-cells can serve as a source of such nucleic acids. The expression cassettes may be inserted into an expression plasmid(s), which is (are) then transfected into host cells, which do not otherwise produce immunoglobulins. Expression is performed in appropriate prokaryotic or eukaryotic host cells and the immunoglobulin is recovered from the cells after lysis or from the culture supernatant.

The term “recombinant mammalian cell” refers to a cell into which a nucleic acid, e.g. encoding a heterologous polypeptide, can be or is introduced/transfected. The term “cell” includes cells which are used for the expression of nucleic acids. In one embodiment the mammalian cell is a CHO cell (e.g. CHO K1, CHO DG44), or a BHK cell, or a NS0 cell, or a SP2/0 cell, or a HEK 293 cell, or a HEK 293 EBNA cell, or a PER.C6® cell, or a COS cells. Especially preferred is a CHO cell, or a BHK cell, or a PER.C6® cell. As used herein, the expression “cell” includes the subject cell and its progeny. Thus, the term “recombinant cell” includes the primary transfected cell and cultures including the progeny cells derived there from without regard to the number of transfers. It is also understood that all progeny may not be precisely identical in DNA content, due to deliberate or inadvertent mutations. Variant progeny that have the same function or biological activity as the originally transformed cell are included.

For the purification of recombinantly produced heterologous immunoglobulins often a combination of different column chromatography steps is employed. Generally a Protein A affinity chromatography is followed by one or two additional separation steps. The final purification step is a so called “polishing step” for the removal of trace impurities and contaminants like aggregated immunoglobulins, residual HCP (host cell protein), DNA (host cell nucleic acid), viruses, or endotoxins. For this polishing step often an anion exchange material in a flow-through mode is used.

Different methods are well established and widespread used for protein recovery and purification, such as affinity chromatography with microbial proteins (e.g. protein A or protein G affinity chromatography), ion exchange chromatography (e.g. cation exchange (carboxymethyl resins), anion exchange (amino ethyl resins) and mixed-mode exchange), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and electrophoretical methods (such as gel electrophoresis, capillary electrophoresis) (Vijayalakshmi, M. A., Appl. Biochem. Biotech. 75 (1998) 93-102).

The following examples and figures are provided to aid the understanding of the present invention, the true scope of which is set forth in the appended claims. It is understood that modifications can be made in the procedures set forth without departing from the spirit of the invention.

### EXAMPLES

### Example 1

### Material and Methods

### Cell Density:

Cell density is measured via the trypan blue staining method. The exclusion of the negatively charged dye trypan blue from viable cells due to their intact cell membrane enables the distinction between viable and non-viable cells (see e.g. Freshney, R. (1987) Culture of Animal Cells: A Manual of Basic Technique, p. 117, Alan R. Liss, Inc., New York.). The method is carried out automatically in a cell culture analyzer CEDEX HiRes (Innovatis AG, Bielefeld, Germany). The viability is then calculated as the ratio of viable cells from the total cell count.

### Substrates and Metabolites:

The concentration of glucose, lactate, ammonia, glutamine and glutamate is measured by enzymatic and ion-selective sensors. The apparatus used is a NOVA BioProfile 100 (NOVA biomedical, Rödermark, Germany).

### Immunoglobulin Concentration:

The concentration of immunoglobulin is measured by affinity chromatography with protein A as specific immunoglobulin binding agent (PorosA column, Applied Biosystems). The elution profile of the immunoglobulin is determined by absorption measurement at a wavelength of 280 nm.

pH, pCO2, pO2:

A blood gas analyzer (pHOx, NOVA biomedical, Rödermark, Germany) is used for the measurement of pH and the partial pressure of oxygen (pO₂) and carbon dioxide (pCO₂).

### Osmolality:

Osmolality is determined by the measurement of freezing point depression in a sample which can be correlated to the content of solutes (Osmomate 030, Gonotec, Berlin, Germany).

### Example 2

### Small Scale Shaker Flask Cultivation—General Proceedings

In a sterile 250 ml Erlenmeyer flask (Corning, Cat.-Nr. 431144) equipped with a ventilation cap with integrated pH- and oxygen-sensor (Precision Sensing GmbH, Regensburg, Germany, Cat.-Nr. 200000919) 120 ml cultivation medium was filled under sterile conditions using a clean bench. The shaker flask containing the cultivation medium was conditioned in an incubator with temperature control which has been validated providing sufficient precision for temperature adjustment. The conditioning of the medium comprises the incubation of the flasks containing a medium not yet inoculated with cells at the temperature, gas pressure conditions and mechanical movement conditions to be used in the beginning of the cultivation. After the conditioning the medium is inoculated with CHO cells expression a recombinant immunoglobulin under sterile conditions using a clean bench. The cultivation is performed as fed-batch-cultivation. During the cultivation the pH- and pO₂-value in the cultivation medium is determined by a chemo-optical fluorescence sensor in situ. The pCO₂ is controlled via the settings of the incubator. Beside the continuous determination of the pH-value, the pO₂-value and the pCO₂-value, the following parameters were determined after every 24 hours of cultivation:
cell density,
substrate (glucose) and metabolite (glutamine, ammonia, glutamic acid, lactic acid, lactate dehydrogenase activity) concentrations,
immunoglobulin concentration in the supernatant,
osmolality.

Based on the determined values of the above listed parameters none, one or more of the following parameters was/were changed once every 24 hours:
the pO₂ by changing the mechanical movement speed,
the pH by changing the outside CO₂ gas pressure in the incubator (acid control) or addition of base (base control),
addition of nutrient solution.

### Example 3

### Small Scale Shaker Flask Cultivation with Constant Mechanical Movement and Constant pCO₂ in the Incubator

An example (preferably monoclonal) antibody for which a small scale cultivation can be performed according to the present invention is an antibody against the amyloid β-A4 peptide (anti-Aβ antibody). Such an antibody and the corresponding nucleic acid sequences are, for example, reported in WO 2003/070760 or US 2005/0169925.

A small scale cultivation of CHO cells expressing an anti-Aβ antibody was performed in a Kühner incubator (Kühner AG, Birsfelden, Switzerland, model B08) with the following parameters:

| parameter | value |
|---|---|
| shaker eccentricity | 5 cm |
| inoculation cell density | 5 × 10⁵ cells/ml |
| starting volume | 120 ml |
| mechanical movement | 160 rpm |
| outside CO₂ gas content | 5% saturation |
| pH | approx. 7 |
| pO₂ | >25% air saturation |
| sample volume | 4.5 ml |

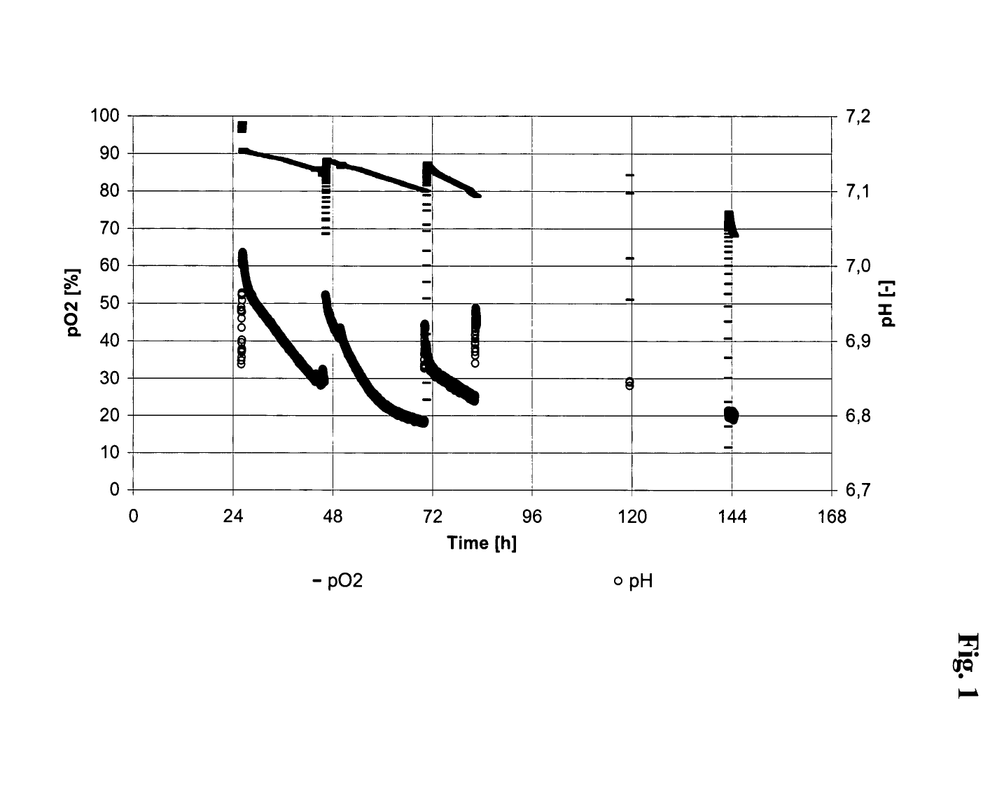

The determined pO₂- and pH-values are shown in FIG. 1. It can be seen from FIG. 1 that the oxygen partial pressure (pO₂) in the solution is very high (above 70% air saturation) and that the oxygen content and pH value of the cultivation medium is rising after each sample drawing.

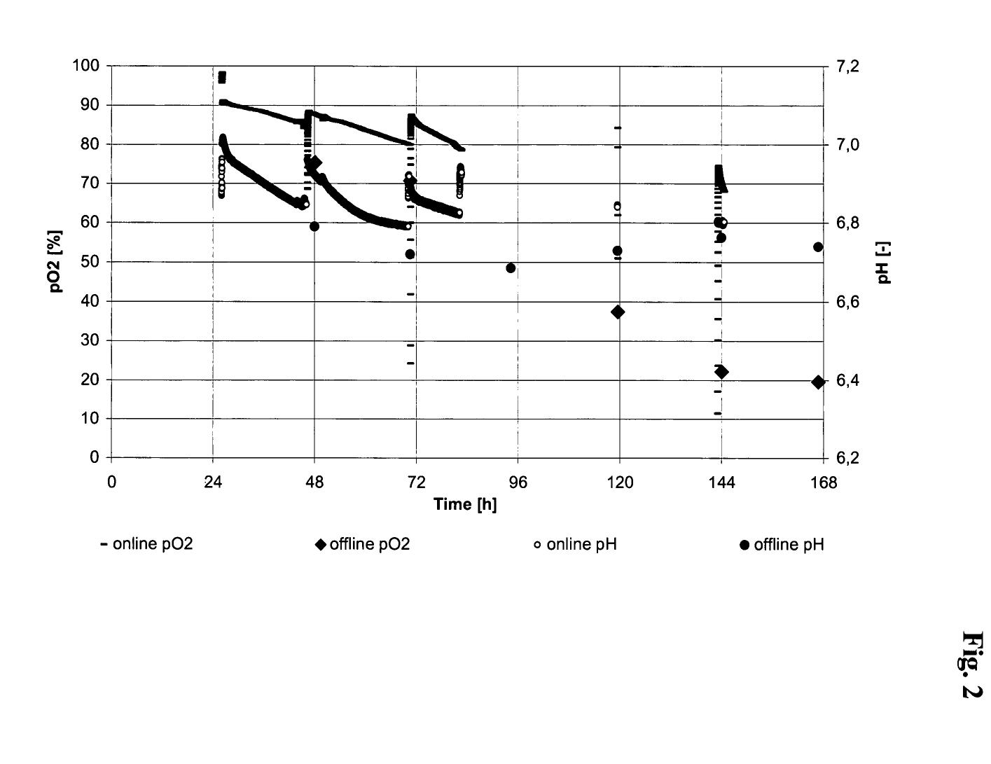

A comparison of the in situ determined values (online) and the values determined from the taken samples (offline) is shown in FIG. 2. It can be concluded:
the pO₂ values determined offline are lower than those determined online;
the offline determined pO₂ is after 144 hours lower than the threshold value of 25% air saturation which indicates a oxygen limitation which is not present in reality;
the pH values determined offline are lower than those determined online;
the difference in the pH value between the online values and the offline values shows no cell density dependency.

### Example 4

### Small Scale Shaker Flask Cultivation with Variable Mechanical Movement and Variable pCO₂ in the Incubator

A small scale cultivation of CHO cells expressing an anti-Aβ antibody was performed in a Kühner incubator (Kühner AG, Birsfelden, Switzerland, model B08) with the following parameters:

| parameter | value |
|---|---|
| shaker eccentricity | 5 cm |
| inoculation cell density | 5 × 10⁵ cells/ml |
| starting volume | 120 ml |
| mechanical movement | variable depending on |
| | online determined pO₂ |
| outside CO₂ gas content | variable depending on |
| | online determined pH |
| pH | approx. 7 |
| pO₂ | >25% air saturation |
| sample volume | 4.5 ml |

The adjustment of the rate of the rotational movement was done according to the following table:

| viable cell density | rate of the rotational |
|---|---|
| [×10⁵ cells/ml] | movement [rpm] |
| 5 | 100 |
| 10 | 120 |
| 20 | 140 |
| 40 | 160 |
| 67.5 | 190 |
| 85 | 200 |
| 100 | 200 |
| 105 | 210 |
| 105 | 150 |
| 105 | 140 |
| 105 | 135 |

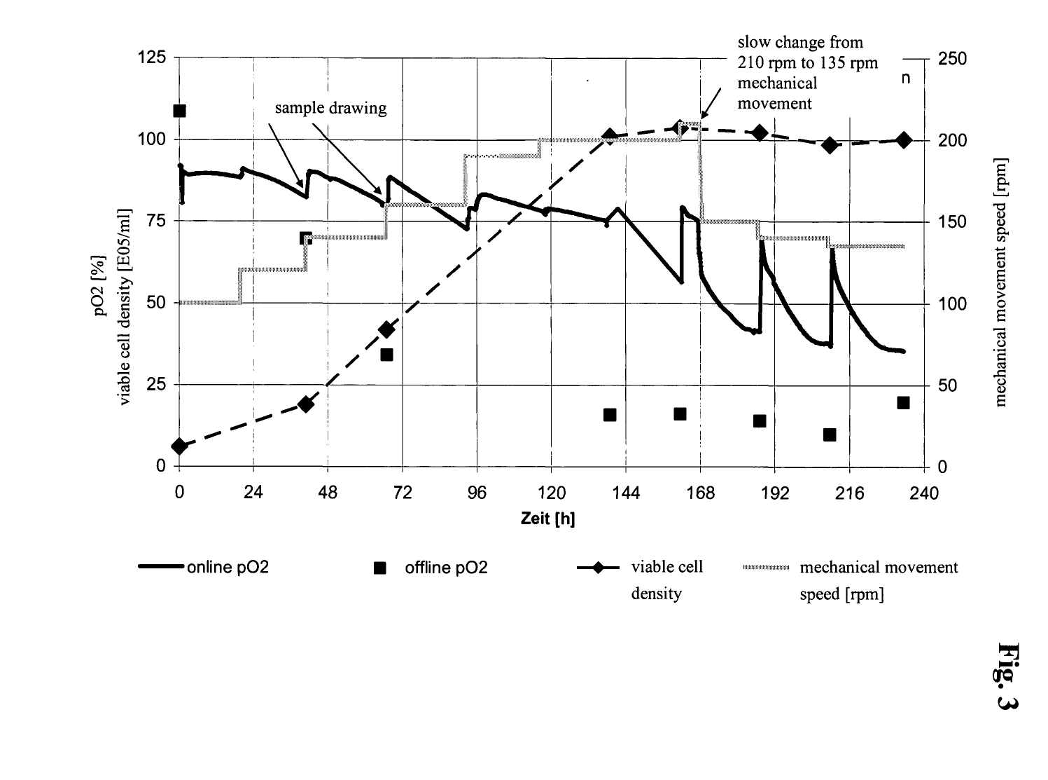

In FIG. 3 are shown the course of online determined pO₂, offline determined pO₂, viable cell density and mechanical movement speed during the cultivation.

The following was observed:
by changing the rate of the rotational movement it is possible to increase or decrease the oxygen partial pressure in the liquid phase;
the short time required between sampling and offline oxygen determination is sufficient at cell densities above 1×10⁶ cells/ml to reduce the oxygen content to below 15% air saturation pointing at an oxygen limitation in the cultivation medium.

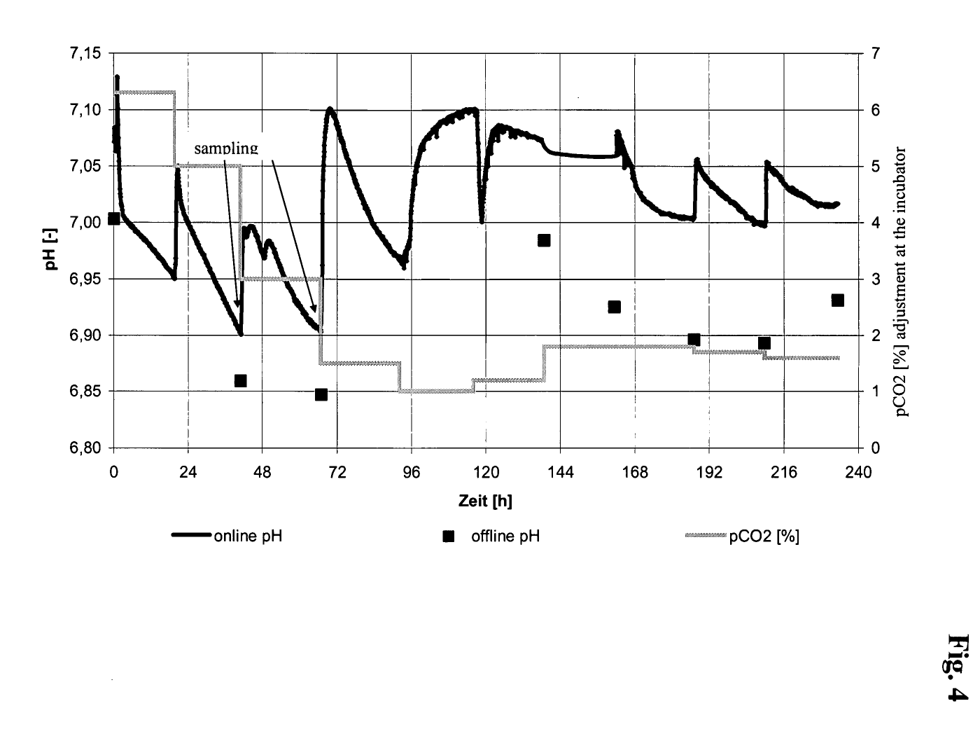

In FIG. 4 are shown the course of online determined pH, offline determined pH, and pCO₂ during the cultivation.

The following was observed:
by changing the outside CO₂ gas pressure in the incubator it is possible to change the pH value in the cultivation medium; a pCO₂ reduction in the incubator results in a pH increase in the cultivation medium;
the online determined pH values are more basic than the offline determined pH values;
due to the conversion of oxygen to carbon dioxide of the cells in the sample the pH value is lowered compared to the cultivation vessel resulting in too low pH values determined offline.

Thus, from the examples shown above it can be concluded that with the offline determination values are obtained that differ from the online determined values and, therefore, would lead to different parameter ranges for later use in a large scale cultivation.

1. Method for the cultivation of a mammalian cell comprising the step of changing the mechanical movement speed of the cultivation vessel depending
i) on the viable cell density in the cultivation medium.

2. Method according to claim 1, characterized in that said changing is further depending
ii) on the oxygen partial pressure in the cultivation medium.

3. Method according to any one of claim 1 or 2, further comprising one or more of the following:
a) cultivating a set of cultivation vessels
each of a total volume of up to 200 μl, or
each with a working volume of up to 100 μl, or
each of a total volume of from 25 ml to 3000 ml, or
each with a working volume of 10 ml to 1500 ml,
b) agitating the cultivation medium by a mechanical movement of the entire cultivation vessel,
c) adjusting the pH value of the cultivation medium via the carbon dioxide concentration in the outside gas phase of the cultivation vessel.

4. Method according to any one of the preceding claims, characterized in that the mechanical movement is by a rotary motion of the entire cultivation vessel or by lulling the entire cultivation vessel or by shaking the entire cultivation vessel.

5. Method according to any one of the preceding claims further comprising
d) determining the pH value and the pO₂ within the cultivation vessel via a non invasive chemo-optical sensor.

6. Method according to any one of the preceding claims, characterized in that the mechanical movement speed is adjusted as follows:
i) setting the mechanical movement speed to 60 rpm to 100 rpm depending on the starting cell density, with
60 rpm at a cell density of 1×10⁵ cells/ml or lower,
80 rpm at a cell density of 2.5×10⁵ cells/ml,
100 rpm at a cell density of 5×10⁵ cells/ml,
or at a linearly intervening value at an intervening cell density,
ii) increasing the mechanical movement speed by 20 rpm for each doubling of the viable cell density up to a cell density of 20×10⁵ cells/ml,
iii) increasing the mechanical movement speed by 20 rpm for each increase of the viable cell density of 20×10⁵ cells/ml up to a cell density of 80×10⁵ cells/ml,
iv) increasing the mechanical movement speed by 10 rpm for an increase of the viable cell density from 80×10⁵ cells/ml to a cell density of 100×10⁵ cells/ml.

7. Method according to claim 6, characterized in that the mechanical movement speed is further adjusted as follows:
v) maintaining the mechanical movement speed at 200 rpm to 210 rpm, reducing the mechanical movement speed stepwise to 135 rpm, and keeping it constant until the cultivation is finished.

8. Method according to claim 7, characterized in that in step v) the mechanical movement speed is maintain at 200 rpm to 210 rpm for between 18 hours and 30 hours.

9. Method according to any one of the preceding claims further comprising
e) increasing the mechanical movement speed to increase the oxygen partial pressure in the cultivation medium or decreasing the mechanical movement speed to lower the oxygen partial pressure in the cultivation medium.

10. Method for the production of a heterologous polypeptide comprising the following steps:
a) providing a mammalian cell comprising a nucleic acid encoding said heterologous polypeptide,
b) cultivating said mammalian cell with a method according to any one of claims 1 to 9,
c) recovering the heterologous polypeptide from the cultivation medium.

11. Method according to claim 10, characterized in that the heterologous polypeptide is an immunoglobulin, or an immunoglobulin fragment, or an immunoglobulin conjugate.

12. Method according to any one of claim 10 or 11, characterized in that the mammalian cell is a CHO cell, a HEK cell, a BHK cell, a NS0 cell, a SP2/0 cell, or a hybridoma cell.

13. Use of a non invasive chemo-optical sensor for the determination of the pH value and the pO₂ in a small scale cultivation vessel in a method according to any one of claims 1 to 9.

14. Use of a method according to any one of claims 1 to 9 for the determination of the cultivation parameter ranges for a large scale cultivation in a stirred cultivation vessel with a volume of 1,000 l to 25,000 l.
